(19) United States
(12) Patent Application Publication
(10) Pub. No.: US 2024/0050679 A1
(43) Pub. Date: Feb. 15, 2024

(54) RIGID ENDOTRACHEAL INTUBATION STYLET

(71) Applicant: Samantha REEDER, Gilbert, AZ (US)

(72) Inventor: Samantha REEDER, Gilbert, AZ (US)

(21) Appl. No.: 18/491,581

(22) Filed: Oct. 20, 2023

Related U.S. Application Data
(63) Continuation-in-part of application No. 17/698,978, filed on Mar. 18, 2022, now abandoned.
(60) Provisional application No. 63/258,169, filed on Apr. 16, 2021.

(51) Int. Cl. A61M 16/04; A61B 1/267

(52) U.S. Cl. CPC A61M 16/0488; A61B 1/267

(57) ABSTRACT

A stylet with a body, a handle, and a tip. The stylet is configured to be inserted into an endotracheal tube. The body is formed of a rigid material and may have a straight section and a curved section. The handle is fixedly coupled to the body and facilitates removal of the stylet from the endotracheal tube. The tip is fixedly coupled to the body and is configured to elastically deform. The tip may have a truncated cone shape. The tip may be angled with respect to the body. When the stylet is fully inserted into the endotracheal tube, the tip of the stylet may extend beyond the end of the endotracheal tube. The stylet may be configured to provide structure to the endotracheal tube and enable use of a laryngoscope during the endotracheal intubation procedure.

### CROSS REFERENCE TO RELATED APPLICATIONS

This application claims the benefit of the filing date of U.S. patent application Ser. No. 17/698,978 entitled “Rigid Endotracheal Intubation Stylet with Extended, Angulated, Tapered, and Pliable Tip” to Samantha Reeder that was filed on Mar. 18, 2022, which application claims priority to U.S. Provisional Patent Application 63/258,169 entitled “Reeder Stylet” to Samantha Reeder that was filed on Apr. 16, 2021, the disclosure of each which are hereby incorporated herein by this reference.

### TECHNICAL FIELD

Aspects of this document relate generally to an endotracheal intubation stylet, and more specifically to a stylet with an extended, angulated, tapered, and pliable tip.

### BACKGROUND

Video endotracheal intubations are performed during procedural and emergent airway management. This procedure aims to establish a definitive airway for a patient to breathe by placing one end of a hollow tube into the trachea and past the glottis while the other end is kept external to the patient. When performing an endotracheal intubation via video laryngoscopy, the medical provider does not always have a clear path for the endotracheal tube to enter the airway. To accomplish this goal, a stylet is inserted inside an endotracheal tube and maneuvered inside a patient's upper airway to position the endotracheal tube to pass through the vocal cords. Once the endotracheal tube is past the vocal cords, the provider removes the stylet, establishing a definitive airway. Stylets currently in use should remain inside the distal portion of the endotracheal tube throughout the procedure and never contact the patient's vocal cords directly.

However, a patient's airway could be obscured or mispositioned in relation to the provider's view, therefore making it difficult or impossible to maneuver the endotracheal tube into the patient's airway. Under these circumstances, the endotracheal tube is effectively blocked from passing the vocal cords and entering the trachea to establish the airway. The angle of entry for the tube can become difficult to reach or the actual opening between the vocal cords too small to penetrate. Current stylets are not designed to make contact with the patient due to the stylet's lack of protrusion from the endotracheal tube. Additionally, if current stylets were to protrude past the endotracheal tube, their composition would result in damage to the trachea. This limitation decreases the success of the procedure and delays critical patient care.

### SUMMARY

Aspects of this document relate to stylet configured to be inserted into an endotracheal tube for an endotracheal intubation procedure, comprising a solid elongated body having a first section adjacent a first end of the body and a second section adjacent a second end of the body distal to the first end, wherein the body is formed of a rigid material, the first section extends straight in a first direction, and the second section is curved such that the second end is oriented in a second direction that is substantially perpendicular to the first direction, a handle fixedly coupled to the first end of the body and configured to facilitate removal of the stylet from the endotracheal tube, and a tip fixedly coupled to the second end of the body and configured to elastically deform, the tip having a truncated cone shape, wherein the tip is angled toward the handle with respect to the second end of the body, wherein when the stylet is fully inserted into the endotracheal tube, the tip of the stylet extends beyond an end of the endotracheal tube, and wherein the stylet is configured to provide structure to the endotracheal tube and enable use of a laryngoscope during the endotracheal intubation procedure.

Particular embodiments may comprise one or more of the following features. The truncated cone shape may be an oblique truncated cone. The tip may have a hardness between 90 and 100 on the Shore A scale. The body may comprise a first material and the tip may comprise a second material. The first material may have a Young's Modulus higher than a Young's Modulus of the second material. An angle between the tip and the body may be between 15 degrees and 45 degrees.

Aspects of this document relate to stylet configured to be inserted into an endotracheal tube for an endotracheal intubation procedure, comprising an elongated body formed of a rigid material and having a solid core, a handle fixedly coupled to a first end of the body and configured to facilitate removal of the stylet from the endotracheal tube, and a tip fixedly coupled to a second end of the body distal to the first end and configured to elastically deform, wherein the tip is angled with respect to the second end of the body, wherein when the stylet is fully inserted into the endotracheal tube, the tip of the stylet extends beyond an end of the endotracheal tube.

Particular embodiments may comprise one or more of the following features. The stylet may be configured to provide structure to the endotracheal tube and enable use of a laryngoscope during the endotracheal intubation procedure. The tip may have a truncated cone shape. The truncated cone shape may be an oblique truncated cone. The body may have a hardness between 60 and 80 on the Shore D scale. The tip may have a hardness between 90 and 100 on the Shore A scale. The body may comprise a first material and the tip may comprise a second material. The first material may have a Young's Modulus higher than a Young's Modulus of the second material. An angle between the tip and the body may be between 15 degrees and 45 degrees.

Aspects of this document relate to stylet configured to be inserted into an endotracheal tube for an endotracheal intubation procedure, comprising an elongated body formed of a rigid material and having a solid core, a handle fixedly coupled to a first end of the body and configured to facilitate removal of the stylet from the endotracheal tube, and a tip fixedly coupled to a second end of the body distal to the first end and configured to elastically deform, wherein the tip extends away from the second end at a fixed oblique angle with respect to the second end and with respect to the first end, wherein when the stylet is fully inserted into the endotracheal tube, the tip of the stylet extends beyond an end of the endotracheal tube, and wherein the stylet is configured to provide structure to the endotracheal tube and enable use of a laryngoscope during the endotracheal intubation procedure.

Particular embodiments may comprise one or more of the following features. A first section of the body adjacent the first end may extend straight in a first direction and a second section of the body adjacent the second end may be curved such that the second end is oriented in a second direction that is substantially perpendicular to the first direction. The tip may have a truncated cone shape. The truncated cone shape may be an oblique truncated cone. The tip may have a hardness between 90 and 100 on the Shore A scale. The body may comprise a first material and the tip may comprise a second material. The first material may have a Young's Modulus higher than a Young's Modulus of the second material. The angle between the tip and the body may be between 15 degrees and 45 degrees.

Aspects of this document relate to a method of performing an endotracheal intubation procedure, comprising inserting a stylet into a lumen of an endotracheal tube, wherein the stylet comprises an elongated body with a handle on a first end of the body and a tip on a second end of the body distal to the first end, extending the tip of the stylet through the endotracheal tube and past an end of the endotracheal tube, introducing the tip of the stylet and the end of the endotracheal tube into an airway of a patient, advancing the stylet and the endotracheal tube into the airway of the patient until the tip of the stylet approaches the glottis of the patient, advancing the tip of the stylet through the glottis, after advancing the tip of the stylet through the glottis, advancing the end of the endotracheal tube through the glottis and into the trachea of the patient, and backing the stylet out of the endotracheal tube.

Particular embodiments may comprise one or more of the following features. The method may comprise dilating the glottis of the patient with the tip of the stylet. The method may comprise before advancing the tip of the stylet through the glottis, inserting a laryngoscope into the airway of the patient adjacent to the endotracheal tube. The tip may have a truncated cone shape. The tip may have a hardness between 90 and 100 on the Shore A scale. The body may comprise a first material and the tip may comprise a second material. The first material may have a Young's Modulus higher than a Young's Modulus of the second material. An angle between the tip and the body may be between 15 degrees and 45 degrees.

The foregoing and other aspects, features, and advantages will be apparent from the specification, drawings, and the claims.

### BRIEF DESCRIPTION OF THE DRAWINGS

Implementations will hereinafter be described in conjunction with the appended drawings, where like designations denote like elements, and:

FIG. 1 is a perspective view of a rigid endotracheal intubation stylet;

FIG. 2 is a front view of the stylet;

FIG. 3 is a back view of the stylet;

FIG. 4 is a right-side view of the stylet;

FIG. 5 is a left-side view of the stylet;

FIG. 6 is a top view of the stylet;

FIG. 7 is a bottom view of the stylet;

FIG. 8 is a close-up view of the angled tip of the stylet, taken from circle 8 in FIG. 2;

FIG. 9 is a perspective view of the stylet inserted into an intubation tube;

FIG. 10 is an exploded view of the stylet and the intubation tube;

FIG. 11 is a close-up view of the tip extending past the end of the intubation tube, taken from circle 11 in FIG. 9;

FIG. 12 is a diagram showing the stylet and intubation tube inserted into a patient's airway;

FIG. 13A is a cross section of the stylet taken from line 13-13 in FIG. 2; and

FIG. 13B is an alternative cross section of the stylet taken from line 13-13 in FIG. 2, showing a core and an outer layer.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of implementations.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

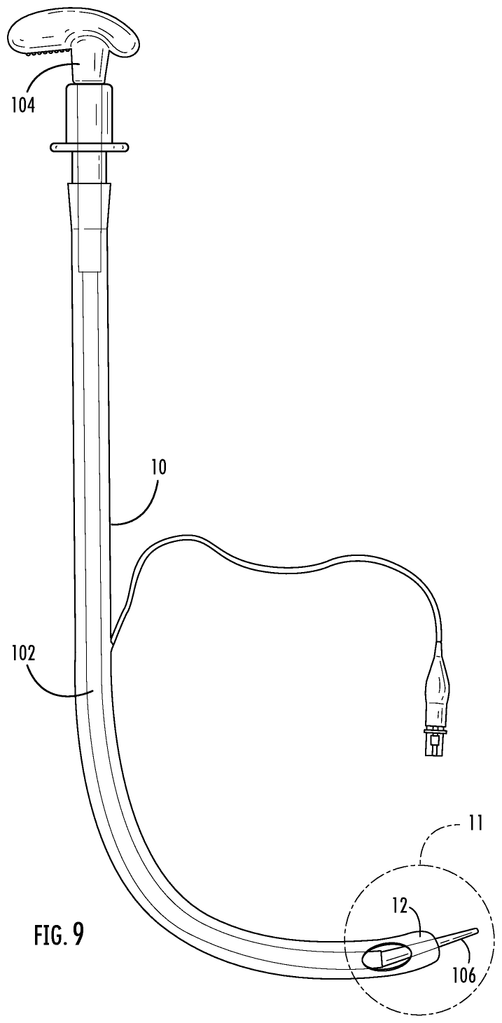

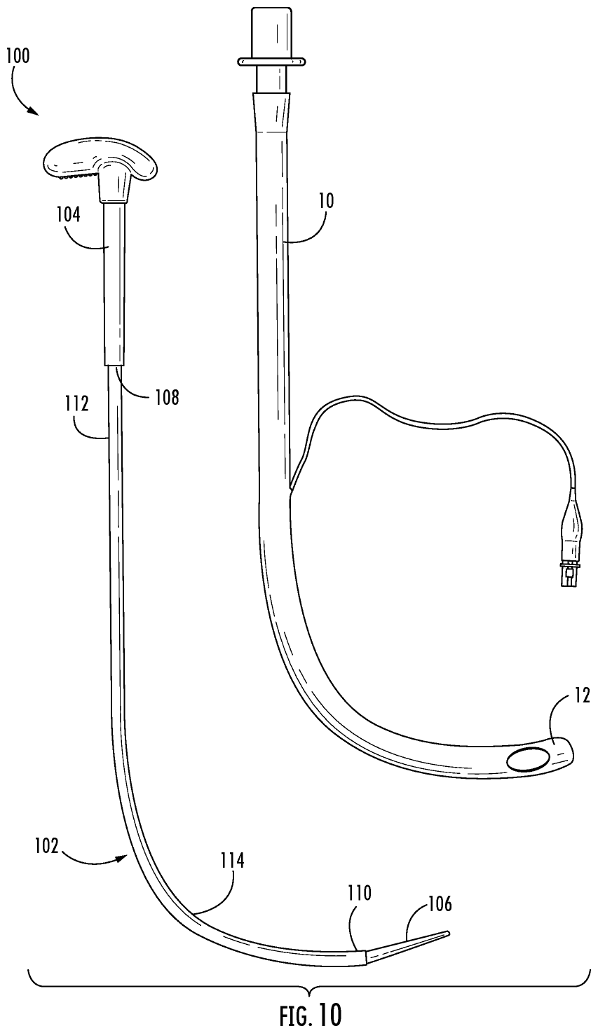

The present disclosure is related to a rigid endotracheal intubation stylet 100. The stylet 100 is configured to insert into the lumen an endotracheal tube 10 for an endotracheal intubation procedure (see FIGS. 9 and 10). During the endotracheal intubation procedure, the stylet 100 is configured to facilitate insertion of the endotracheal tube 10 into the patient's trachea and/or increase the first-pass success rate of the procedure. The stylet 100 is particularly useful when using video laryngoscopy. Video laryngoscopy involves inserting a laryngoscope into the patient's airway along with the endotracheal tube 10. During a video laryngoscopy, the patient's airway is not opened completely, preventing a good view of the trachea and the vocal cords. Because the airway is still partially collapsed, the stylet 100 must be rigid enough to help direct the tube 10 through the airway without bending in the airway. Thus, the stylet 100 must be more rigid than is typical. In this way, the stylet 100 is configured to provide structure to the endotracheal tube 10 and enable use of a laryngoscope during the endotracheal intubation procedure.

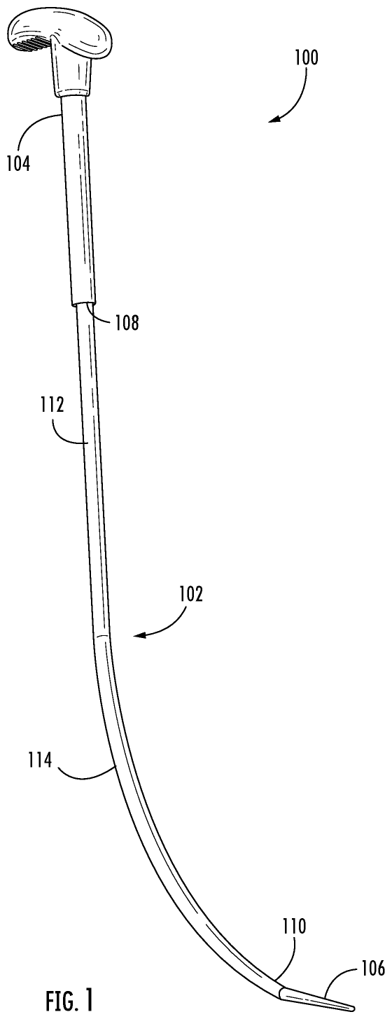

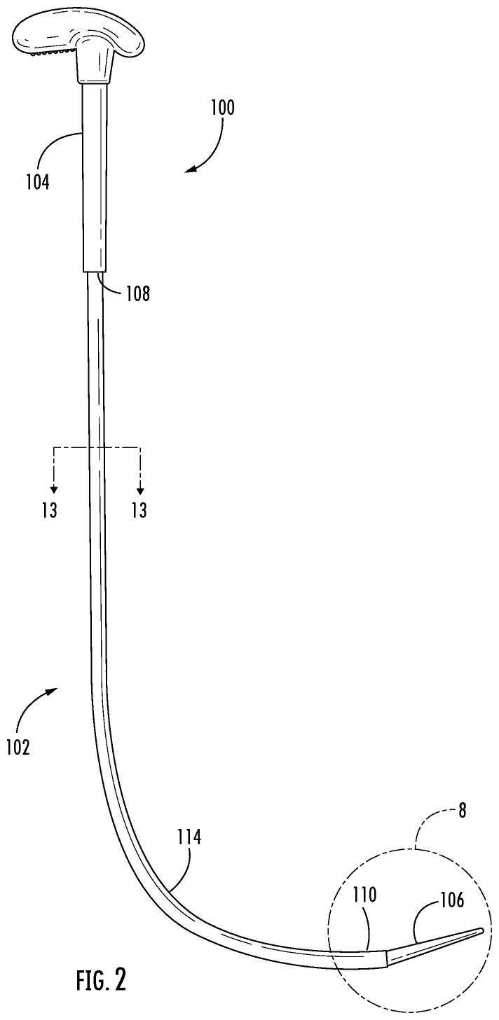

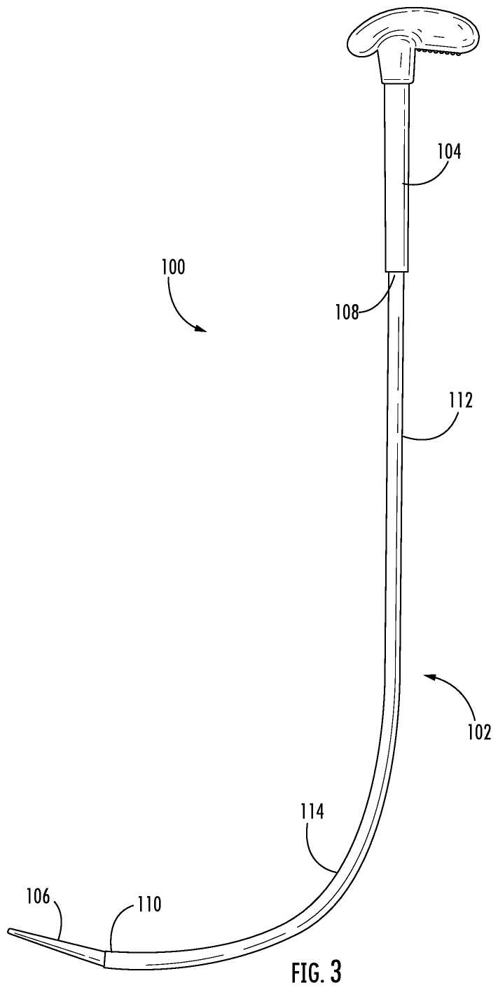

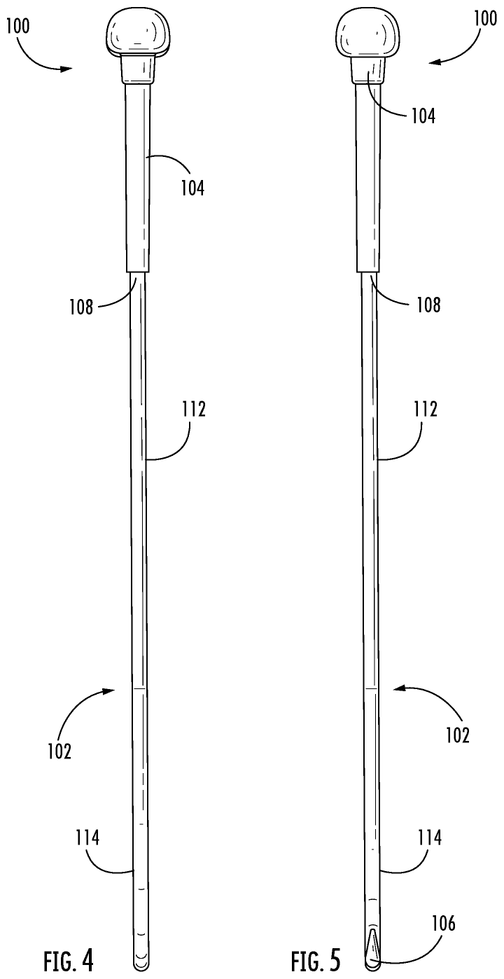

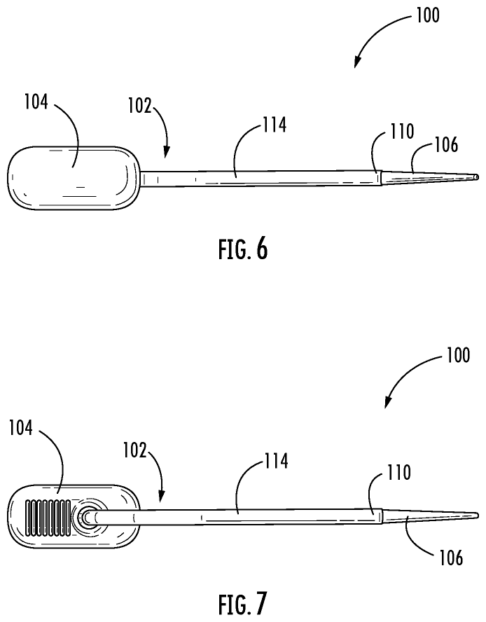

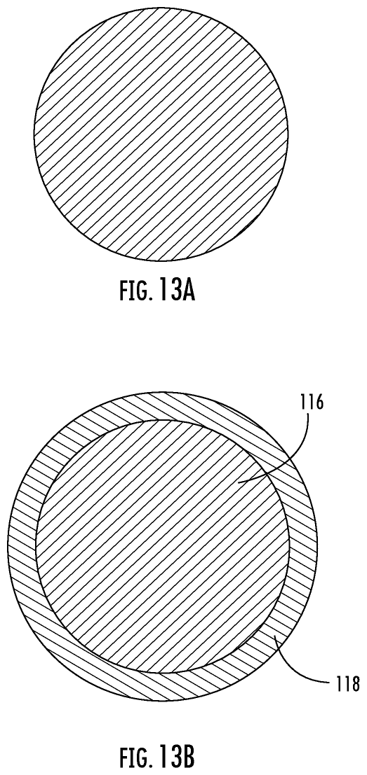

The stylet 100 may comprise a body 102, a handle 104, and a tip 106, as shown in FIGS. 1-8. The body 102 may be elongated to allow the body 102 to be inserted into the endotracheal tube 10 and into the patient's airway. The body 102 may also be solid (see FIG. 13A), with no hollow core. This helps to increase the rigidity of the body 102 to provide the needed support as discussed above. Additionally, the body 102 may be formed of a rigid material and/or a material with a high Young's Modulus to increase the rigidity of the body 102. For example, the body 102 may be formed of metal or have a core 116 formed of metal (see FIG. 13B). In some embodiments, the body comprises a material with a hardness between 60 and 80 on the Shore D scale. The body 102 may comprise materials commonly used in 3D printing, such as Polylatic Acid (PLA) or Acrylonitrile Butadiene Styrene (ABS), and may be 3D printed.

In some embodiments, the body 102 is wrapped in a secondary material (see FIG. 13B). In such embodiments, the rigidity of the body 102 may be provided by the core 116 and the outer layer 118 may help to join the body 102 and the tip 106 together.

The body 102 may have a first end 108 and a second end 110 distal to the first end 108. The body 102 may also have a first section 112 adjacent to the first end 108 and a second section 114 adjacent to the second end 110. In some embodiments, the first section 112 extends straight in a first direction, and in particular embodiments, the second section 114 is curved. The second section 114 may be curved such that the second end 110 is oriented in a second direction that is substantially perpendicular to the first direction. In other words, the curve of the second section 114 may turn the direction of the body 102 by about 90 degrees. Two ends that are substantially perpendicular may not be exactly perpendicular. For the purposes of this disclosure, the terms “substantially perpendicular” and “about 90 degrees” are meant to include embodiments where the angles are within 15 degrees of being 90 degrees or within 15 degrees of being perpendicular. For example, in an embodiment that has a second section 114 that is curved such that the second end 110 is oriented in a second direction that forms an angle of 75 degrees with the first direction of the first end 108, the first direction and the second direction are still considered to be substantially perpendicular to each other.

The handle 104 is fixedly coupled to the first end 108 of the body 102. The handle 104 is configured to facilitate removal of the stylet 100 from the endotracheal tube 10. This is important because, once the tube 10 is placed in the patient's airway, the stylet 100 needs to be removed to allow the tube 10 to be used to deliver air to the patient. The handle 104 acts as a stop on the stylet 100 to prevent the stylet 100 from inserting too far into the tub 10 and becoming unremovable. For example, the handle 104 may be larger than the body 102 such that the body 102 fits into the tube 10 and at least a portion of the handle 104 does not, as shown in FIG. 9, so that the handle is always accessible from outside the tube 10. The handle 104 is also formed to provide a more ergonomic way to handle the stylet 100 than simply gripping a thin rod.

Returning to FIGS. 1-8, the tip 106 is fixedly coupled to the second end 110 of the body 102. The tip 106 may be bonded to the body 102 with a silicone glue or may be fastened to the body 102 with a locking screw. As mentioned above, in some embodiments, the body 102 has a core 116 and an outer layer 118. In such embodiments, the outer layer 118 may be formed of the same material as the tip 106 and thus the tip 106 and the outer layer 118 may be integrally formed together. For example, the tip 106 and the outer later 118 may be formed of silicone and may be formed as a single piece, with the body 102 encased in the silicone. In such embodiments, the tip 106 may be less prone to separation from the body 102.

The tip 106 is an important feature of the stylet 100 because the tip 106 is designed to probe forward within the patient's airway without damaging the soft tissue of the airway. Thus, the tip 106 may be formed of a softer material. For example, the tip 106 may comprise a material with a hardness between 90 and 100 on the Shore A scale. The tip 106 may comprise a material with a hardness of 95 on the Shore A scale. The tip 106 is configured to be more flexible than the body 102. This may be accomplished by selecting a material with a lower Young's Modulus, as well as by forming the tip 106 in a shape that is more flexible, such as by decreasing the cross section of the tip 106.

The tip 106 is configured to elastically deform so that the tip 106 retains its shape when no force is applied but bends when a force is applied. In particular because the body 102 must be rigid as discussed above and the tip 106 must be more flexible, the difference in flexibility and/or rigidity of these two components is important. Thus, in particular embodiments, the body 102 comprises a first material and the tip 106 comprises a second material, where the first material has a Young's Modulus that is higher than a Young's Modulus of the second material. For example, the first material may be a stainless steel and the second material may be silicone. As another example, the first material may be a 3d printed material that is rigid, such as Polylatic Acid (PLA) or Acrylonitrile Butadiene Styrene (ABS), and the second material may be silicone. In both these examples, the Young's Modulus of the first material is higher than the Young's Modulus of the second material.

The tip 106 may be tapered. For example, the tip 106 may have a cone shape or a truncated cone shape. In some embodiments, the cone shape or truncated cone shape that is used is oblique, meaning that the axis of the cone is not perpendicular to the base of the cone, or that the apex of the cone is not centered over the base of the cone. Any other shape may also be used for the tip 106. In embodiments with a tapered tip 106, the tip 106 acts to dilate the vocal cords and provide an accessible opening for the endotracheal tube 10.

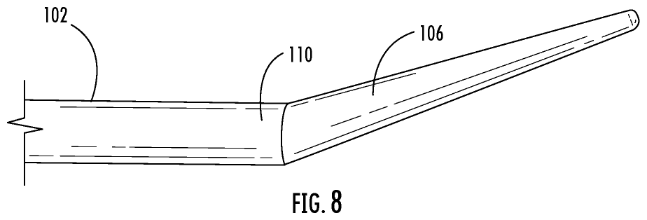

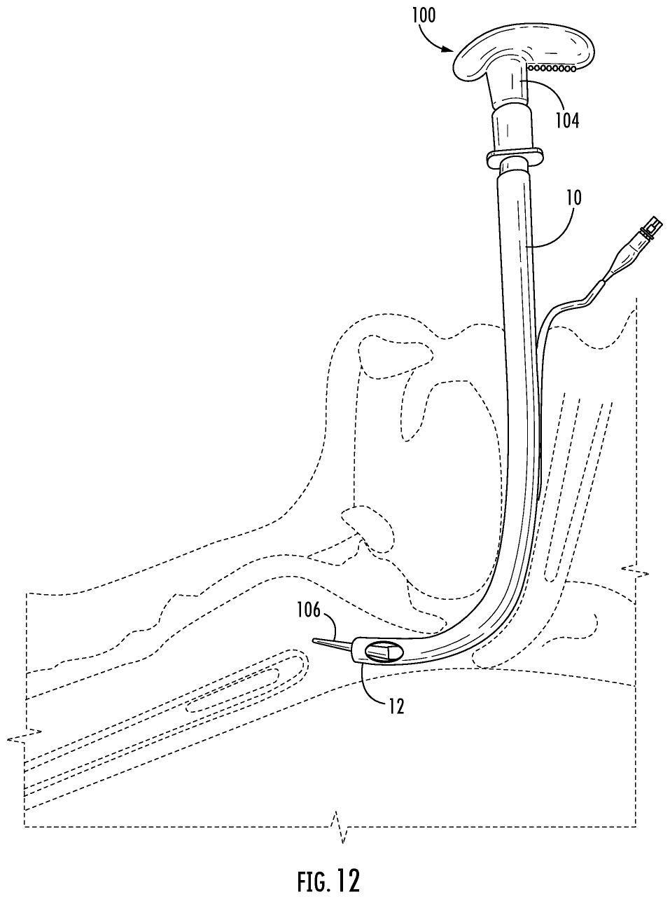

The endotracheal tube 10 must be inserted through the trachea of the patient to for a successful endotracheal intubation procedure. The trachea is positioned anteriorly in the airway, with the esophagus behind it, as can be seen in FIG. 12. Thus, the tip 106 may be angled with respect to the second end 114 of the body 102. In some embodiments, the tip 106 is angled toward the handle 104, as shown in FIG. 8. Because the tip 106 is fixedly coupled to the body 102, this angle is constant and/or fixed, aside from flexing that occurs in the tip 106 when a force is applied. By having an angle between the tip 106 and the second end 110, the tip 106 is lifted anteriorly within the patient toward the trachea when the stylet 100 is inserted into the patient's airway. This facilitates the endotracheal intubation procedure by automatically lifting the tip 106 up towards the trachea, rather than requiring the medical professional performing the procedure to manipulate the stylet in a way that brings the tip up towards the trachea. In other words, angulation of the tip 106 decreases the amount of manipulation needed to direct the endotracheal tube 10 because the tip 106 is in an anatomically beneficial position by being better aligned with the patient's airway. The angle between the tip 106 and the body 102 may be between 15 degrees and 45 degrees. In particular embodiments, the angle is between 25 degrees and 35 degrees. The angle between the tip 106 and the body 102 may be 30 degrees.

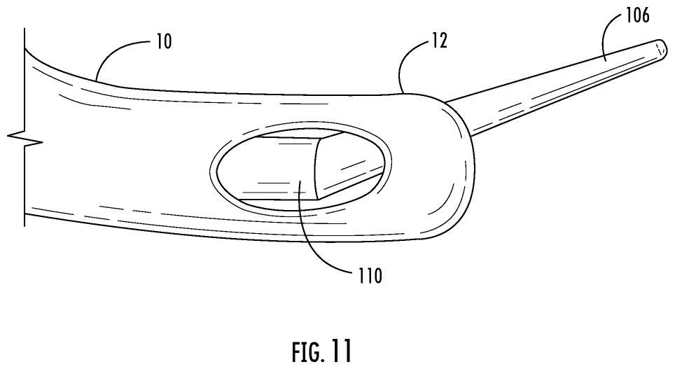

In typical stylets, the tip does not extend past an end 12 of the tube 10 so that the stylet does not injure the patient. On the other hand, when the stylet 100 is fully inserted into the endotracheal tube 10, the tip 106 of the stylet 100 extends beyond the end 12 of the endotracheal tube 10, as shown in FIG. 11. This allows the tip 106 to guide the tube 10 where it needs to go. Because the tip 106 is flexible, the tip 106 can make contact with the wall of the patient's airway without causing injury. In some embodiments, the tip 106 extends between one and four centimeters past the end 12 of the tube 10.

Thus, the stylet 100 solves the problem of difficult video endotracheal tube intubation by providing a rigid stylet 100 with a flexible and protruding tip 106. The rigid body 102 of the stylet 100 is inserted into the endotracheal tube 10, but the tip 106 of the stylet 100 protrudes from the tube 10. The extended tip 106 is pliable so it may pass through and make contact with the vocal cords without harming the trachea. Additionally, angulation of the tip 106 decreases the amount of manipulation needed to direct the endotracheal tube 10 because the tip 106 is in an anatomically beneficial position by being better aligned with the patient's airway. The tapered, pliable tip 106 acts to dilate the vocal cords and provide an accessible opening for the endotracheal tube 10. Once the tip 106 is past the vocal cords, the endotracheal tube 10 is advanced along the elongated tip 106 of the stylet 100 directly into the trachea to establish the airway.

In performing the endotracheal intubation procedure with the stylet 100, a healthcare provider will insert the stylet 100 into the lumen of an endotracheal tube 10 so that the tip 106 of the stylet 100 protrudes between 1 and 4 or between 2 and 3 centimeters past the end 12 of the tube 10. The pliable tip 106 is the only portion of the stylet 100 that should protrude from endotracheal tube 10 opposite the handle 104 of the stylet 100. Once assembled in this fashion, the healthcare provider will hold the tube 10 and stylet 100 so that the handle 104 is external to the patient and the tip 106 is entering the oropharynx. Posterior to the epiglottis, the tube 10 and stylet 100 navigate toward the patient's vocal cords. The combined rigidity of the stylet 100 and the pliability of the tip 106 grants the medical provider maneuverability in an airway while minimizing concern of perforating tracheal walls. Additionally, because of its composition and protrusion from the tube 10, the tip 106 will act as a hyper angulated director that is thinner than the endotracheal tube 10 to be able to turn anteriorly and slip through the vocal cords to easily direct the tube 10 into the trachea, as shown in FIG. 12. The pliable, tapered tip 106 of the stylet 100 is then used to pass the vocal cords, dilating the cords as the tip 106 and tube 10 are advanced. Once the tip 106 is successfully past the vocal cords, the endotracheal tube 10 advances into the trachea to be secured and the stylet 100 is backed out of the tube 10 by the handle 104.

Thus, a method of performing an endotracheal intubation procedure may comprise inserting the stylet 100 into the lumen of an endotracheal tube 10. As discussed above, the stylet 100 may have an elongated body 102 with a handle 104 on a first end 108 of the body 102 and a tip 106 on a second end 110 of the body 102. The method may also comprise extending the tip 106 of the stylet 100 through the endotracheal tube 10 and past an end 12 of the endotracheal tube 10. The tip 106 of the stylet 100, as well as the end 12 of the endotracheal tube 10 may be introduced into the airway of the patient, and the stylet 100 and the endotracheal tube 10 may be advanced into the airway of the patient until the tip 106 of the stylet 100 approaches the glottis of the patient. Before advancing the tip 106 of the stylet 100 through the glottis, a laryngoscope may be inserted into the airway of the patient adjacent to the endotracheal tube 100. The method may comprise advancing the tip 106 of the stylet 100 through the glottis. This may comprise dilating the glottis of the patient with the tip 106 of the stylet 100. After advancing the tip 106 of the stylet 100 through the glottis, the method may comprise advancing the end 12 of the endotracheal tube 10 through the glottis and into the trachea of the patient. The method may also comprise backing the stylet 100 out of the endotracheal tube 10.

It will be understood that implementations of a rigid endotracheal intubation stylet are not limited to the specific assemblies, devices and components disclosed in this document, as virtually any assemblies, devices and components consistent with the intended operation of a rigid endotracheal intubation stylet may be used. Accordingly, for example, although particular rigid endotracheal intubation stylets, and other assemblies, devices and components are disclosed, such may include any shape, size, style, type, model, version, class, measurement, concentration, material, weight, quantity, and/or the like consistent with the intended operation of rigid endotracheal intubation stylets. Implementations are not limited to uses of any specific assemblies, devices and components; provided that the assemblies, devices and components selected are consistent with the intended operation of a rigid endotracheal intubation stylet.

Accordingly, the components defining any rigid endotracheal intubation stylet may be formed of any of many different types of materials or combinations thereof that can readily be formed into shaped objects provided that the materials selected are consistent with the intended operation of a rigid endotracheal intubation stylet. For example, the components may be formed of: polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; glasses (such as quartz glass), carbon-fiber, aramid-fiber, any combination thereof, and/or other like materials; composites and/or other like materials; metals, such as zinc, magnesium, titanium, copper, lead, iron, steel, carbon steel, alloy steel, tool steel, stainless steel, brass, nickel, tin, antimony, pure aluminum, 1100 aluminum, aluminum alloy, any combination thereof, and/or other like materials; alloys, such as aluminum alloy, titanium alloy, magnesium alloy, copper alloy, any combination thereof, and/or other like materials; any other suitable material; and/or any combination of the foregoing thereof. In instances where a part, component, feature, or element is governed by a standard, rule, code, or other requirement, the part may be made in accordance with, and to comply under such standard, rule, code, or other requirement.

Various rigid endotracheal intubation stylets may be manufactured using conventional procedures as added to and improved upon through the procedures described here. Some components defining a rigid endotracheal intubation stylet may be manufactured simultaneously and integrally joined with one another, while other components may be purchased pre-manufactured or manufactured separately and then assembled with the integral components. Various implementations may be manufactured using conventional procedures as added to and improved upon through the procedures described here.

Accordingly, manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, a fastener (e.g. a bolt, a nut, a screw, a nail, a rivet, a pin, and/or the like), wiring, any combination thereof, and/or the like for example, depending on, among other considerations, the particular material forming the components.

It will be understood that methods for manufacturing or assembling rigid endotracheal intubation stylets are not limited to the specific order of steps as disclosed in this document. Any steps or sequence of steps of the assembly of a rigid endotracheal intubation stylet indicated herein are given as examples of possible steps or sequence of steps and not as limitations, since various assembly processes and sequences of steps may be used to assemble rigid endotracheal intubation stylets.

The implementations of a rigid endotracheal intubation stylet described are by way of example or explanation and not by way of limitation. Rather, any description relating to the foregoing is for the exemplary purposes of this disclosure, and implementations may also be used with similar results for a variety of other applications employing a rigid endotracheal intubation stylet.

What is claimed is:

1. A stylet configured to be inserted into an endotracheal tube for an endotracheal intubation procedure, comprising:
a solid elongated body having a first section adjacent a first end of the body and a second section adjacent a second end of the body distal to the first end, wherein the body is formed of a rigid material, the first section extends straight in a first direction, and the second section is curved such that the second end is oriented in a second direction that is substantially perpendicular to the first direction;
a handle fixedly coupled to the first end of the body and configured to facilitate removal of the stylet from the endotracheal tube; and
a tip fixedly coupled to the second end of the body and configured to elastically deform, the tip having a truncated cone shape, wherein the tip is angled toward the handle with respect to the second end of the body;
wherein when the stylet is fully inserted into the endotracheal tube, the tip of the stylet extends beyond an end of the endotracheal tube; and
wherein the stylet is configured to provide structure to the endotracheal tube and enable use of a laryngoscope during the endotracheal intubation procedure.

2. The stylet of claim 1, wherein the truncated cone shape is an oblique truncated cone.

3. The stylet of claim 1, wherein the tip has a hardness between 90 and 100 on the Shore A scale.

4. The stylet of claim 1, wherein the body comprises a first material and the tip comprises a second material and wherein the first material has a Young's Modulus higher than a Young's Modulus of the second material.

5. The stylet of claim 1, wherein an angle between the tip and the body is between 15 degrees and 45 degrees.

6. A stylet configured to be inserted into an endotracheal tube for an endotracheal intubation procedure, comprising:
an elongated body formed of a rigid material and having a solid core;
a handle fixedly coupled to a first end of the body and configured to facilitate removal of the stylet from the endotracheal tube; and
a tip fixedly coupled to a second end of the body distal to the first end and configured to elastically deform, wherein the tip is angled with respect to the second end of the body;
wherein when the stylet is fully inserted into the endotracheal tube, the tip of the stylet extends beyond an end of the endotracheal tube.

7. The stylet of claim 6, wherein the stylet is configured to provide structure to the endotracheal tube and enable use of a laryngoscope during the endotracheal intubation procedure.

8. The stylet of claim 6, wherein the tip has a truncated cone shape.

9. The stylet of claim 8, wherein the truncated cone shape is an oblique truncated cone.

10. The stylet of claim 6, wherein the body has a hardness between 60 and 80 on the Shore D scale.

11. The stylet of claim 6, wherein the tip has a hardness between 90 and 100 on the Shore A scale.

12. The stylet of claim 6, wherein the body comprises a first material and the tip comprises a second material and wherein the first material has a Young's Modulus higher than a Young's Modulus of the second material.

13. The stylet of claim 6, wherein an angle between the tip and the body is between 15 degrees and 45 degrees.

14. A method of performing an endotracheal intubation procedure, comprising:
inserting a stylet into a lumen of an endotracheal tube, wherein the stylet comprises an elongated body with a handle on a first end of the body and a tip on a second end of the body distal to the first end;
extending the tip of the stylet through the endotracheal tube and past an end of the endotracheal tube;
introducing the tip of the stylet and the end of the endotracheal tube into an airway of a patient;
advancing the stylet and the endotracheal tube into the airway of the patient until the tip of the stylet approaches the glottis of the patient;
advancing the tip of the stylet through the glottis;
after advancing the tip of the stylet through the glottis, advancing the end of the endotracheal tube through the glottis and into the trachea of the patient; and
backing the stylet out of the endotracheal tube.

15. The method of claim 14, further comprising dilating the glottis of the patient with the tip of the stylet.

16. The method of claim 14, further comprising, before advancing the tip of the stylet through the glottis, inserting a laryngoscope into the airway of the patient adjacent to the endotracheal tube.

17. The method of claim 14, wherein the tip has a truncated cone shape.

18. The method of claim 14, wherein the tip has a hardness between 90 and 100 on the Shore A scale.

19. The method of claim 14, wherein the body comprises a first material and the tip comprises a second material and wherein the first material has a Young's Modulus higher than a Young's Modulus of the second material.

20. The method of claim 14, wherein an angle between the tip and the body is between 15 degrees and 45 degrees.
